(19) United States
(12) Patent Application Publication
(10) Pub. No.: US 2010/0093048 A1
(43) Pub. Date: Apr. 15, 2010

(54) Process and Apparatus for Assisting the Extraction and Processing of Biodiesel Oil Using Oil-Bearing and Other Organic Feedstock

(76) Inventor: Richard M. Marshall, Lake Country (CA)

Correspondence Address: Anthony C Edwards, P.O. Box 26020, Westbank, British Columbia, V4T 2G3, CA

(21) Appl. No.: 12/451,389

(22) PCT Filed: May 12, 2008
(86) PCT No.: PCT/CA2008/000878

§ 371 Date: Nov. 10, 2009

Related U.S. Application Data
(60) Provisional application No. 60/924,358, filed on May 10, 2007.

(51) Int. Cl. C12P 5/02; C12M 1/00

(52) U.S. Cl. 435/167; 435/289.1

(57) ABSTRACT

A process and apparatus for assisting the extraction and processing of biodiesel oil from organic feedstock includes: providing crushed oil-bearing organic feedstock meal from which has been extracted a first amount of biodiesel oil wherein the meal retains a second amount of entrained oil; forming a meal slurry containing said meal and passing the slurry to an anaerobic digester; anaerobically digesting the meal slurry so as to convert said second amount of entrained oil to produce heat, methane gas, and organic fertilizer or oil-free cattle feed; providing an electrical generator and employing the methane gas for at least the production of electricity by burning the methane gas in the electrical generator which is adapted to convert heat to electricity and re-cycling at least some of the electricity.

### CROSS REFERENCE TO RELATED APPLICATION

This application claims priority from U.S. Provisional Patent Application No. 60/924,358 filed May 10, 2007 entitled Method and Apparatus for Producing Biodiesel Fuel.

### FIELD OF THE INVENTION

This invention relates to the field of production of bio-diesel fuel and in particular to a process and apparatus for assisting the extraction and processing of biodiesel oil using oil-bearing and other organic feedstock.

### BACKGROUND OF THE INVENTION

The biodiesel process was developed many years ago and the original engines built by Rudolph Diesel were designed to operate on biodiesel oil made from peanut oil. Only recently has there been significant interest in the production of biodiesel oil. This growth has been spurred on due to rapidly increasing hydro-carbon oil and refinery production costs combined with a push towards the use of more environmentally friendly fuels. Biodiesel oil fits the need quite well but it also has inherent processing issues which introduce production costs and environmental issues that are holding back the production of biodiesel oil.

Though some biodiesel processes are highly sophisticated and technologically advanced, the basics of biodiesel production from Canola, Soy and animal fats is simple. Biodiesel production does however have some inherent difficulties that have greatly restricted the development and profitability of manufacturing biodiesel, and have limited the development of the industry as a whole. Most often the production of biodiesel takes place in a facility which is owned and operated by farm co-ops, grain companies and small enterprises, which most often do not have technical and operational expertise readily available. The biodiesel process must therefore be much less complex than processes such as for Ethanol production, while at the same time, producing significantly efficient results. Developers of the biodiesel process, and the industry as a whole, need to address the following issues: oil extraction, energy consumption, process waste, integration as a part of the community, and feedstock costs.

With respect to the oil extraction issue, to process Canola and other oil crops, it is advantageous that the entrained oils be removed through a process which uses as little energy as possible while also removing as much of the oil as possible. The crushing of canola through steam injection and pressing is straightforward, but often up to 15% of the total canola oil can still be left in the waste meal, also referred to herein as crushed oil-bearing feedstock meal. To extract this difficult remaining 15%, a process of solvent extraction is often used in the prior art. Solvent extraction processes are very labor-intensive, polluting, dangerous, troublesome and often have a high capital cost. This additional process is not something which most owners of a biodiesel facility would want to have, but leaving 15% oil in the meal means the full energy value, and therefore the full economic value, is not being extracted from, for example, the Canola.

### SUMMARY OF THE INVENTION

The present invention uses anaerobic digestion in an anaerobic digestion plant to process the high oil meal slurry from a biodiesel process to eliminate the solvent extraction portion of the process. The left over products will be processed into fertilizers. Mustard or mustard seed can be added to the process to increase the natural pesticide attributes of the fertilizer. In addition, the glycerin byproduct from the initial biodiesel extraction and processing plant can also be processed in the anaerobic digestion plant to aid in increasing biogas production. The process takes the biogas from the anaerobic digester and produces electricity through a biogas burning generator or produces natural gas through a scrubbing and compression process to be sold back to the public gas utility supply or bottled.

The primary issues are; the energy required in the facility; and the environmental issues related to the prior art solvent extraction of the oil from the grain meal after the seeds have been processed. With the addition of an anaerobic digestion (AD) facility, the solvent extraction facility can be eliminated and the meal and the retained oil can be processed into biogas for natural gas or power generation. This solution eliminates the environmentally damaging issues surrounding solvent extraction while simultaneously removing the risks related to increasing energy costs through the generation and use of the energy produced from the AD process.

The next issue inherent in the production of biodiesel is the generation of large amounts of glycerin which ordinarily require disposal or further processing into a value added product. Present pricing and the outlook for supplementary markets is very poor and the glycerin is presenting a difficult issue for biodiesel producers. In the process according to the present invention the glycerin may be added to the AD process to increase the biogas and in turn the energy production of the process.

To retain the simplicity of a biodiesel process while extracting optimum value, the process according to the present invention employs anaerobic digestion, combined with complimentary ancillary technologies. This process changes the overall operation of a biodiesel facility to use waste and low valued products in such a way, as to reduce operational risk, while producing additional value added products which increase facility revenues.

The process employs the use of all waste meal coming from the crush facility with the extra oils entrained, in such a way that it is digested to produce large volumes of heat, methane gas and organic fertilizer. The methane gas would be used for two purposes; for the production of electricity through burning of the methane gas in a generator, and to be scrubbed to natural gas quality for injection into a natural gas system to be used as a direct source or bottled fuel. The meal can be utilized as a fertilizer but also as an oil free cattle feed if the meal is not combined with other organic materials, such as manure or other wastes not consisting of pure organics.

To do this will involve the construction of a process according to the present invention to handle the waste discharge of canola (or other oil crops) coming from the crushing facility. The process according to the present invention will preheat the slurry coming into the facility to 52° C., where it will be held in a retention tank and constantly agitated while having water added, to produce a 13 to 15% solids slurry, that once conditioned, will be pumped into the anaerobic digester vessel to begin the methogenic process.

With the process according to the present invention, the retained energy in the processed (crushed) canola meal can be extracted and converted to valuable methane gas, which can be used as “Natural Gas” quality gas, or it can be used in a generator to produce electricity. As anaerobic digestion is well known around the world and is undemanding to operate, it is a perfect addition to the biodiesel process in order to effectively remove and convert the energy retained in the canola waste, into useable or marketable energy. The electricity produced by the generators can be used to operate the facility, and the excess production can be sold back to the grid.

With respect to the energy consumption issue, with the rapidly increasing cost of energy, the total benefit of employing energy to create biodiesel to be used as energy has been significantly reduced. The biodiesel industry requires increased operational efficiencies as well as “green” methods of producing and using energy to remain a true environmental option.

The heat created from the stack gases of the electrical generator can be salvaged through the use of a “Heat Recovery Steam Generator” (HRSG). The HRSG unit will have multiple taps, which will allow for hot water, as well as steam to be produced for the different uses within a biodiesel facility. One such use is to heat the incoming feedstocks to the required operational temperature prior to being added to the digesters. As the Anaerobic Digesters operate, the reaction is exothermic and additional heat is created. In the process according to the present invention this heat is applied as a heat source for an integrated greenhouse facility. The excess warm and nutrient rich water is used to fertilize the plants, and the stack gas CO₂ collected is injected into the greenhouse air to accelerate plant growth. The waste products from the greenhouse facility can be re-introduced into the process to further increase the production of methane gas and additional energy.

In addition to the biodiesel process requirements, the process according to the present invention produces excess electrical power and natural gas, which can be sold as “Green” products for a preferred price. As energy costs go down, the amount of energy used internally in the facility can be increased. As energy prices rise, more energy can be sold to the grid. This provides the facility with an internal hedging strategy to mitigate the energy risks.

With respect to the process waste issue, as with most production processes there remains waste streams, which must be dealt with. These products are not easily disposed of, and have the potential to reduce the profitability of the facility. The biodiesel facility where the feedstock is initially crushed to extract and process the 85% or so biodiesel (the “first amount of biodiesel oil” referred to herein) will produce meal and glycerin as waste products. These at one time had value as a cattle feed and a glycerin food additive. With the massive increase in the production of biodiesel and oil crops, there is a severe “glut” on the markets, and glycerin and meal often do not have sufficient inherent value to merit the processing and transportation costs required to make them useable products. Therefore they need to be dealt with in a manner advantageous to the process.

With the process according to the present invention, these waste products undergo the natural conversion of glycerin and oils to energy in the form of methane gas. The remaining meal solids are processed through pelletizing and drying, into a value added organic fertilizer. Often, as with the case of adding greenhouse wastes, the different feedstocks provide beneficial characteristics to the resulting organic fertilizer. This fertilizer can be custom blended and sold in bulk, or it can be reconstituted to make up specific fertilizers with varying nitrogen, potassium, and phosphate ratings. The liquid stream can also be concentrated further and sold for horticultural use. This gives the owner/operator of the Biodiesel facility the ability to create future added products with significant value which are in great demand. With no waste or greenhouse gas emissions, the process according to the present invention provides the ability to have an extremely small environmental footprint with the net ability to be a provider of CO₂ credits.

The process according to the present invention process uses the lowest amount of water of any Biodiesel process on the market. In fact, all of the water used in the facility is recycled through the anaerobic digestion system and ultra filtration system and reintroduced as makeup water for the process.

With respect to the integration as a part of the community issues construction of any industrial based facility comes with challenges and demands which will alter the community in which it is constructed. Every process should account for this, and offer flexibility which can accommodate the combined needs, so that the process is not only placed into a community, but integrated as a positive component in the lives of the residents. Scaled energy facilities will often, and understandably so, generate significant opposition that can be overcome with planning and design procedures which integrate the facility into the community in a synergistic approach, which benefits not only the proponents of the project, but also the community.

The process according to the present invention has the ability to integrate wastes such as organics and cattle waste. Often the digested meal solids can be pelletized as animal feed and the wastes from the cattle used in the anaerobic digestion plant to create energy.

The process according to the present invention allows for bioenergy production with minimal impact. The process operates without smell, process discharges, excessive noise or pollution. The environmental permitting is minimal, as there are no discharges. Water requirement for the process of the present invention is also minimal as all water is recycled back into the process. This allows for the process to have somewhat negligible impact on community resources.

In addition to processing waste meal and glycerin from the biodiesel extraction and processing plant, the process can also integrate the addition of other organic sources a community may have. For example, restaurants, food processors, greenhouses, households, and yards may dispose of wastes at the plant operating the process according to the present invention, and these organics may be used to generate additional energy. This energy may in turn be used in the community grids as natural gas or electricity.

The process according to the present invention may provide one or more of Fossil Fuel Neutral and Energy Self Sufficient production, no need for outside energy sources, 100% water recycling, Solutions for Glycerin production, Organic fertilizer production, CO₂ Production, Energy in the form of heat, gas, and electrical, Solutions to economic loss potential from traditional biodiesel technologies, Low environmental footprint generating large volumes of GHG credits, 100% extraction of oil energy from the meal, additional profit centers; greenhouse, CO₂, Gas, Electricity, ability to expand process to accommodate other feedstocks from the community, high value cattle feeds.

The process according to the present invention provides environmentally friendly and low cost production of biodiesel and bioenergy, with stability through internal generation of energy and processing of profitable alternatives to glycerin and meal production. The production of the cattle feed also becomes an output which may provide revenues to the facility.

In summary, the process and apparatus for assisting the extraction and processing of biodiesel oil using organic and other feedstock according to one aspect of the present invention may be characterized as including:
1) providing crushed oil-bearing organic feedstock meal from which has been extracted a first amount of biodiesel oil wherein the meal retains a second amount of entrained oil;
2) forming a meal slurry containing said meal and passing the slurry to an anaerobic digester;
3) anaerobically digesting the meal slurry so as to convert said second amount of entrained oil to produce heat, methane gas, and organic fertilizer or oil-free cattle feed;
4) providing an electrical generator and employing the methane gas for at least the production of electricity by burning the methane gas in the electrical generator which is adapted to convert heat to electricity and re-cycling at least some of the electricity for use at least in part in at least one of steps 1-3 above.

Advantageously, the step of forming the meal slurry includes holding and heating the slurry, for example in a slurry tank. Forming the slurry may include the step of adding water while agitating the slurry so as to form a meal slurry having solids substantially in the range of 13% to 15% solids, and while maintaining the slurry at a temperature of at least substantially 40 degrees Celsius or higher depending on the feedstock as would be known to one skilled in the art, and wherein the step of passing the slurry to the anaerobic digester includes pumping the slurry from the tank into the anaerobic digester so as to begin a methogenic process in the digester. For example, the slurry may be heated to substantially 52 degrees Celsius.

In one preferred embodiment, warm and nutrient rich waste water and carbon dioxide are formed as a waste product, and wherein the waste water and carbon dioxide are used as a fertilizer for plants. For example, the plants may be grown in a greenhouse process and wherein the greenhouse process produces greenhouse waste products, and wherein the process further comprises the steps of recycling the greenhouse waste products into the anaerobic digester.

In one aspect of the present invention, the electricity from the generator is at least in part sold for use by consumers of electricity, and wherein said step of recycling the electricity further comprises the step of increasing a quantum of the recycled electricity as the price at which the electricity may be sold to consumers of electricity falls, and wherein the step of recycling the electricity further comprises the step of decreasing said quantum of recycled electricity as the price rises.

In the extraction and processing of biodiesel process, waste products, which include meal, and which further include glycerin, are produced end products from crushing of the oil-bearing feedstock during said extraction and processing of the biodiesel oil. The process according to the present invention may further comprise the step of converting said glycerin to methane gas by introducing the glycerine into the meal slurry for anaerobic digestion in the digester.

In a further aspect, the process of the present invention further includes the step of providing a pre-preparation chamber for the pre-preparation and addition of other organic feedstock, for example so as to tailor the food value of the end products for particular uses, to the slurry and to aid in digestion of the meal and to aid in digestion of the glycerin, where the process further includes the steps of heating and chopping said organic feedstock in said pre-preparation chamber and then passing the organic feedstock into shiny. For example, the organic feedstock may be a food value additive feedstock chosen from the group comprising agricultural crops or animal waste, for adding food value to products of said digester for use as animal feed.

In yet a further aspect of the present invention, the process further includes the step of recovering at least some of the heat produced by the generator for use in the heating of the slurry. For example, a heat exchanger may be used in cooperation with said tank, and, where a heat steam recovery generator is provided cooperating with the generator for producing steam, and the steam may be used in the heat exchanger to heat the slurry.

### BRIEF DESCRIPTION OF THE DRAWINGS

FIG. 1 is a diagrammatic view of the operation of a plant using the process according to one embodiment of the present invention.

FIG. 2 is a perspective view of one rotating biological contactor forming one part of the apparatus according to one aspect of the present invention.

FIG. 3 is a diagrammatic view of a prior art zeolite adsorption column or “Z”-cell.

### DETAILED DESCRIPTION OF EMBODIMENTS OF THE INVENTION

The process according to the present invention is an integrated process which uses waste meal 12 and glycerin 14 produced by a biodiesel extraction and processing facility 10 such that the net energy and waste handling requirements of the biodiesel facility, including its associated crush plant, are greatly reduced. The process may combine additional value added feedstocks to produce a value added cattle feed 16.

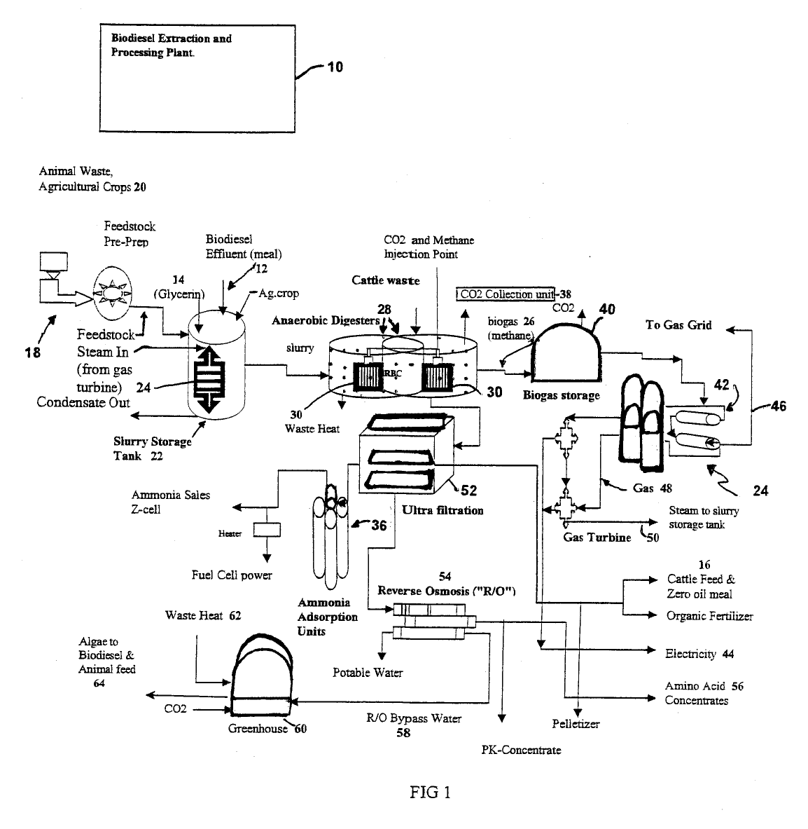

As seen in FIG. 1, the basic process according to the present invention may be depicted as a flow chart of steps as described below.

The feedstock “pre-prep” step is a process using an integrated heating and chopping chamber 18 for the addition into the process of many additional feedstocks 20 which will assist in the digestion of the glycerin and meal. For animal feed applications, these additional feedstocks will not only enable the digestion of the glycerin and meal, they must also add food value to the resultant product. Animal waste from dairy and beef cattle and other animals, as well as household and food processing organic waste can be introduced through the pre-prep process chamber 18 and will be used as an additional feedstock to produce biogas and fertilizer as end products as described below. This pre-prep process through mechanical and the thermal action breaks apart the fibrous materials and, which combined with the heating of the slurry in the next step to Mesophilic (40° C.) and thermophilic (52° C.) temperatures provides for a biological reaction to occur once added to the anaerobic digester in the next following step after a retention time in the Slurry Storage tank.

The slurry storage tank 22 is a food-grade vessel, for example, an insulated stainless steel vessel, which is heated by a set of heat exchangers 24 mounted on the side of the slurry vessel. The slurry is pumped through the heat exchanger and heated by steam on the jacket side. The process steam is generated from a Heat Steam Recovery Generator (HSRG) in the exhaust stack of the electrical generator 24 described below, and as would be known to one skilled in the art. The generator 24 is operated using the biogas 26 produced from the anaerobic digester 28. The heating fluid in the HSRG may be made up of a thermally conductive food grade chemical to avoid contamination of the bio-process. The associated temperature control loop (not shown) may employ automatic valves which modulate the process heating temperature as required as would be known to a skilled workman in the art. The heat exchanger may operate with steam loads or with hot water.

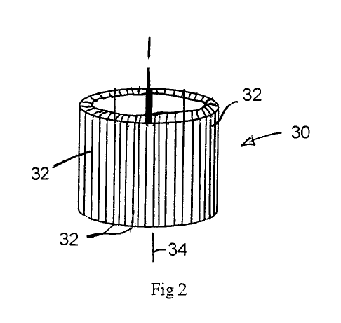

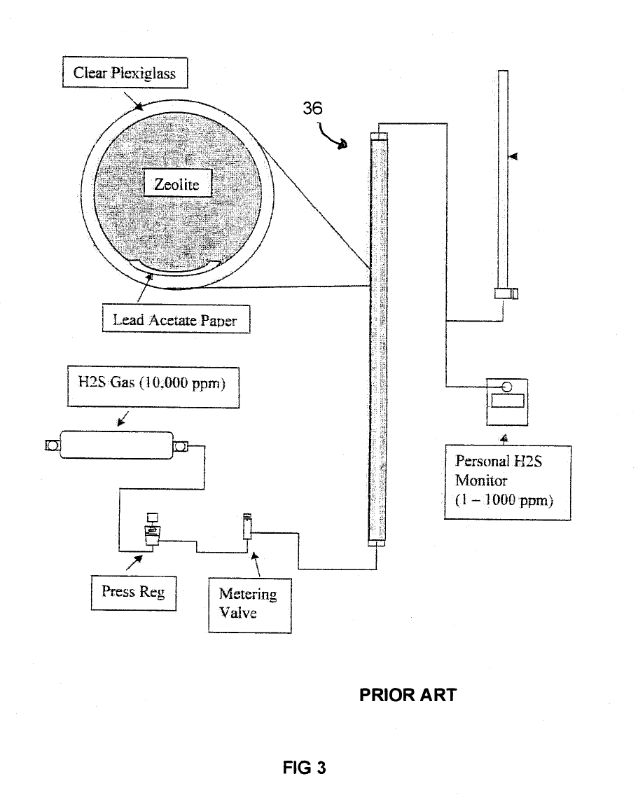

The anaerobic digestion process occurs in an anaerobic digester (AD) 28. Biogas, that is methane (approximately 65-70%), CO₂ (approximately 10%), and other gas in smaller amounts, is generated in the AD 28. Biogas from AD 28 may in one embodiment be re-cycled back into AD 28 by a gas injection system through multiple injection apertures within a Rotating Biological Contactor (RBC) 30 such as seen in FIG. 2. The RBC 30 is a rotating mechanical agitator which increases the contact surface of the bacteria seeding process and allows for increased biogas yield and shorter digestion times. The RBC may be of Polyurethane and have fins 32 so that as it rotates slowly about a vertical axis 34 within AD 28 it provides a biological bonding surface area and simultaneous injection of CO₂ and methane. The RBC agitates the slurry to keep the solids suspended. The biogas and other gases produced by AD 28 are directed through an ammonia adsorption column 36 as known in the prior art and depicted by way of example in FIG. 3 (“Z”-cell) as required to control ammonia concentrations to ensure optimum digestion.

The carbon dioxide (CO₂) collection system consists of a filtration unit, membrane separation (scrubbing) and compression systems which although not illustrated are well know in the art. The process collects the CO₂ created by AD 28 in a CO₂ collection unit 38 for transportation and sale in a raw form. Some of this CO₂ may be added to the makeup air in the greenhouse, and another stream of CO₂ may be used for injection into an algae growth process to assist in the propagation of cells and net output of algae. As stated above, CO₂ may also be pumped through orifices in the RBC 30 and into the AD 28 tank and slurry.

The biogas 26 may be stored in a collapsible gas storage dome 40. The stored gas provides a volume of gas to supply process backup as required and serves as a buffer vessel between the gas production and the scrubbing system.

The gas filtration system 42 may be original equipment manufacturer (OEM) equipment used to clean the gas to pipeline quality natural gas 46, and pressurize for pipeline injection. The process also turns out a low pressure and lower quality gas 48 to be burned by the generator 24 to produce electricity 44 for recycling back into the process, and for sale to utilities depending on the price at which the electricity may be sold back to the electrical grid.

The heat energy will be collected as required, to be used in the process. This is accomplished by using a prior art Heat Recovery Steam Generator (HRSG) with the option of just recovering stack gas heat or being fired by the biogas to provide high quality heat or low pressure steam 50, as required by the process.

The ultra-filtration unit 52 separates the micro-solids from the process slurry. This process allows the slurry to be bypassed for the extraction of the ammonia in the adsorption columns 36, to prevent high levels which hinder the anaerobic digestion process. Ultra-filtration may be accomplished using prior art commercially available equipment such as sold by Monlan Group (www.monlanaroup.com) or Siemens under the trademark MEMCOR (www.water.siemens.com).

Adsorption columns 36 such as sold by N.E.A.T. Environment Inc. (www.neatenvironment.com) use zeolite adsorption to remove the ammonia from the slurry to make the solids more useful as a fertilizer or cattle feed. The columns consist of multiple rechargeable “Z”-cells which are exchanged during operation and new cells replaced. The use of adsorption columns provides for reduced energy consumption by the plant while increasing the quality of the fertilizer produced.

The process may employ an OEM Reverse Osmosis (R/O) system 54 which performs final cleaning of the slurry. The fines retentate may be sold to a fertilizer company for processing into Amino Acid concentrates 56, for horticultural use. The resulting water 58 may be re-introduced as makeup water for the process.

The greenhouse 60 may employ waste heat 62 from the process to provide warm water for processing, as well as ambient heat. The greenhouse may provide vegetables and other products to the community, and any resulting wastes may be added to the anaerobic slurry vessel for further biogas creation.

The greenhouse may also grow algae 64 in a micro control process, and produce considerable volumes of accessible bio-oil. This oil is pressed from the algae, and used in the biodiesel process, or it may be introduced directly into the anaerobic digester for increased gas production.

As an example of the operation of the process of the present invention, a 10 MWH electrical generation plant will require 35 Million m³ of biogas/year of 60-65% CH4 (methane). If only Sorguhm were used as a feedstock to the anaerobic digester this would require that each day 450 tonnes/day would need to be fed into the plant to produce the 10 MWH of power.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A process for assisting the extraction and processing of biodiesel oil from organic feedstock includes the steps of:
1) providing crushed oil-bearing organic feedstock meal from which has been extracted a first amount of biodiesel oil wherein said meal retains a second amount of entrained oil;
2) forming a meal slurry containing said meal and passing said slurry to an anaerobic digester;
3) anaerobically digesting said meal slurry so as to convert said second amount of entrained oil to produce heat, methane gas, and organic fertilizer or oil-free cattle feed;
4) employing said methane gas for at least the production of electricity by burning said methane gas in an electrical generator which is adapted to convert heat to electricity and re-cycling at least some of said electricity for use at least in part in at least one of steps 1-3.

2. The process of claim 1 wherein said step of forming said meal slurry includes holding and heating said slurry in a tank.

3. The process of claim 2 further comprising the step of adding water while agitating said slurry so as to form a meal slurry having substantially 13% to 15% solids at a temperature of at least substantially 40 degrees Celsius, and wherein said step of passing said slurry includes pumping said slurry from said tank into said anaerobic digester so as to begin a methogenic process in said digester.

4. The process of claim 3 wherein said slurry is heated to substantially 52 degrees Celsius.

5. The process of claim 2 wherein warm and nutrient rich waste water is formed as a waste product, and wherein said waste water is used as a fertilizer for plants.

6. The process of claim 5 wherein carbon dioxide gas is formed as a waste product and wherein said carbon dioxide gas is used as a growth accelerator for said plants.

7. The process of claim 6 wherein said plants are grown in a greenhouse process and wherein said greenhouse process produces greenhouse waste products, and wherein said process further comprises the steps of recycling said greenhouse waste products into said anaerobic digester.

8. The process of claim 1 wherein said electricity is at least in part sold for use by consumers of electricity, and wherein said step of recycling said electricity further comprises the step of increasing a quantum of said recycled electricity as the price at which said electricity may be sold to consumers of electricity falls, and wherein said step of recycling said electricity further comprises the step of decreasing said quantum of recycled electricity as said price rises.

9. The process of claim 1 wherein waste products, which include said meal, and which further include glycerin, are produced end products from crushing of said oil-bearing feedstock during said extraction and processing of said biodiesel oil, and wherein the process further comprises the step of converting said glycerin to methane gas by introducing the glycerine into said meal slurry for said anaerobic digestion in said digester.

10. The process of claim 1 further comprising the step of providing a pre-preparation chamber for the pre-preparation and addition of organic feedstock to said slurry to aid in digestion of said meal and to aid in digestion of said glycerin, said process further comprising the steps of heating and chopping said organic feedstock in said pre-preparation chamber and then passing said organic feedstock into said slurry.

11. The process of claim 10 wherein said organic feedstock is a food value additive feedstock chosen from the group comprising agricultural crops or animal waste, for adding food value to products of said digester for use as animal feed.

12. The process of claim 2 wherein said generator produces heat, and wherein said process further comprises the step of recovering at least some of said heat for use in said heating of said slurry.

13. The process of claim 12 further comprising the step of providing a heat exchanger cooperating with said tank, and providing a heat steam recovery generator cooperating with said generator for producing steam, and using said steam in said heat exchanger to heat said slurry.

14. An apparatus for assisting the extraction and processing of biodiesel oil from organic feedstock includes:
1) means for providing crushed oil-bearing organic feedstock meal from which has been extracted a first amount of biodiesel oil wherein said meal retains a second amount of entrained oil;
2) means for forming a meal slurry containing said meal and passing said slurry to an anaerobic digester;
3) means for anaerobically digesting said meal slurry so as to convert said second amount of entrained oil to produce heat, methane gas, and organic fertilizer or oil-free cattle feed;
4) means for employing said methane gas for at least the production of electricity by burning said methane gas in an electrical generator which is adapted to convert heat to electricity and re-cycling at least some of said electricity for use at least in part in at least one of steps 1-3.

15. The apparatus of claim 14 further comprising a slurry tank and means for holding and heating said slurry in said tank.

16. The apparatus of claim 15 further comprising means for adding water to, and means for agitating, said slurry so as to form a meal slurry having substantially 13% to 15% solids, and wherein said means for heating said slurry includes means for heating said slurry to a temperature of at least substantially 40 degrees Celsius.

17. The apparatus of claim 16 wherein warm and nutrient rich waste water is formed as a waste product, and further comprising means for using said waste water as a fertilizer for plants.

18. The apparatus of claim 17 wherein carbon dioxide gas is formed as a waste product and further comprising means for using said carbon dioxide gas as a growth accelerator for said plants.

19. The apparatus of claim 18 further comprising a greenhouse wherein said plants are grown in said greenhouse and wherein said greenhouse process produces greenhouse waste products, and further comprising means for recycling said greenhouse waste products into said anaerobic digester.

20. The apparatus of claim 14 wherein said extraction and processing of biodiesel oil produces glycerin as a waste product, the apparatus further comprising a pre-preparation chamber for the pre-preparation and addition of organic feedstock to said slurry to aid in digestion of said meal and to aid in digestion of said glycerin in said slurry wherein said chamber further includes means for heating and chopping said organic feedstock in said pre-preparation chamber and then passing said organic feedstock into said slurry.

21. The apparatus of claim 15 wherein said generator produces heat, and wherein said apparatus further comprises means for recovering at least some of said heat cooperating with said means for heating of said slurry.

22. The apparatus of claim 21 wherein said means for heating said slurry includes a heat exchanger cooperating with said tank, and wherein said means for recovering at least some of said heat includes a heat steam recovery generator cooperating with said generator for producing steam, and wherein said steam is used in said heat exchanger to heat said slurry.
